(19) United States
(12) Patent Application Publication
(10) Pub. No.: US 2006/0124443 A1
(43) Pub. Date: Jun. 15, 2006

(54) Control and monitoring of non-resonant radiation-induced nucleation, crystallization, and polymorph formation

(76) Inventors: David Tuschel, Monroeville, PA (US); Arjun Bangalore, Monroeville, PA (US)

Correspondence Address: DUANE MORRIS, LLP;IP DEPARTMENT, 30 SOUTH 17TH STREET, PHILADELPHIA, PA, 19103-4196, US

(21) Appl. No.: 11/252,414

(22) Filed: Oct. 18, 2005

Related U.S. Application Data
(60) Provisional application No. 60/625,014, filed on Nov. 3, 2004.

(51) Int. Cl. C07C 41/00; C07C 45/00

(52) U.S. Cl. 204/157.92; 204/157.93

(57) ABSTRACT

The invention relates to methods of assessing the polymorphic form of a substance by assessing Raman-shifted radiation scattered by a particle of the substance. The method is useful, for example, for assessing particle sizes and size distributions in mixtures containing both particles of the substance and other materials. The invention also relates to methods of selecting and controlling polymorph formation by illuminating a material with non-resonant (i.e., non-absorbed) laser radiation as it is thermally driven through a phase transition temperature.

### CROSS-REFERENCES TO RELATED APPLICATIONS

This application is entitled to priority pursuant to 35 U.S.C. §119(e) to U.S. provisional patent application 60/625,014, which was filed on 3 Nov. 2004.

### BACKGROUND OF THE INVENTION

The invention relates generally to the field of compositional analysis of solid particles.

Many chemical compounds can exist in multiple discrete crystalline forms. For example, graphite and diamond are discrete crystalline forms of elemental carbon. The property of being able to assume multiple crystalline forms is commonly designated polymorphism, and the different crystalline forms of the same compound are designated polymorphic forms or, more simply, polymorphs. Polymorphs of a single compound generally have chemical properties that vary in at least subtle ways. For instance, polymorphs can exhibit differences in melting points, electrical conductivities, patterns of radiation absorption, x-ray diffraction patterns, crystal shapes, dissolution rates, and solubilities, even though the polymorphs are made up of the same chemical.

In the context of pharmaceutically active compounds, differences among polymorphs can affect the pharmacological properties of the compound in significant ways. By way of example, the dissolution rate of a drug can greatly influence the rate and extent of bioavailability of the drug when administered by a selected route. Furthermore, the shelf stability of a drug compound can vary significantly, depending on the polymorphic form the drug assumes. In the U.S. and elsewhere, regulatory approval of a drug formulation often requires knowledge and description of the polymorphic form(s) of the drug that occur in the composition submitted for approval. This is so because approvability of a drug substance requires reproducibility in manufacture, dosing, and pharmacokinetic behavior of the drug. In the absence of such reproducibility, safety and efficacy of the drug cannot be sufficiently assured.

The polymorphic form(s) of a compound that are present in a composition is important in other industries as well. By way of example, the properties of dyes and of explosives can be strongly influenced by polymorphism. The crystalline form(s) present in a food product can affect the taste, mouth feel, and other properties of the product.

The crystal shape that a chemical compound assumes can be heavily influenced by the polymorphic form assumed by the compound. In turn, the bulk properties of a preparation of a compound in crystalline form(s) depend on the polymorphic form(s) assumed by the compound in the preparation. For instance, the flow characteristics, tensile strength, compressibility, and density of a powdered form of a compound will be determined by the polymorphs present in the preparation.

Various techniques are known for investigation of polymorphic forms of a compound that occur in the solid state. Such methods include polarized light microscopy (including hot-stage microscopy), infrared spectrophotometry, single-crystal X-ray and X-ray powder diffraction, thermal analysis, and dilatometry. In many instances, these methods can be limited by resolution of the method, polymorphic non-homogeneity of the analyte, similarity among polymorphs of the property analyzed, or other practical difficulties. In particular, compositions that contain multiple polymorphic forms of a compound can be difficult or impossible to analyze using such techniques.

Owing to the properties of a compound in different polymorphic forms, it is often desirable to produce one polymorphic form in preference to (or even substantially to the exclusion of) one or more other polymorphic forms. Under conditions in which multiple polymorphic forms of a compound can be crystallized, the most stable (generally, the least soluble and highest-melting) polymorph will tend to be formed at a greater rate than the other polymorph(s) that can be crystallized under those conditions. However, the rate of formation of crystals of a single polymorphic form can depend heavily on the concentration of crystallization nuclei for that form.

It is common to add pre-existing crystals of a desired polymorph of a compound to a solution from which the compound is to be crystallized, in order to enhance formation of the desired polymorph. However, this method is often unreliable for a given crystallization process, and is ineffective in others. Others have disclosed use of infrared (e.g., U.S. Pat. No. 5,976,325) or polarized light (e.g., U.S. Pat. No. 6,759,521) to enhance crystal nucleation or to influence selective formation of one polymorph over another, both of which methods involve crystallization from solution. Such methods are not universally useful, are restricted to precipitation from solution, and their usefulness can depend on the compound to be crystallized and the reagents and conditions used in the crystallization process.

Under certain conditions, compounds are capable of changing among polymorphic forms, the rate of interchange often depending on the conditions. By way of example, a pharmaceutically active compound can change from one form to another during storage in a container, and the rate of change can vary, depending on the storage conditions. A purchaser of the container will often not know the storage history of the container, and will be unable to determine, or even estimate, the degree to which such interchange may have occurred. The same difficulties encountered during identification of the polymorphic form(s) of a compound inhibit analysis of the degree to which a compound has undergone change from one polymorphic form to another.

Improved methods for assessing the polymorphic form of a compound, particularly in a solid particulate form and methods for influencing the polymorphic form assumed by a compound could overcome or limit the shortcomings identified above. The present invention is related to such methods.

### BRIEF SUMMARY OF THE INVENTION

The invention relates to a method of selecting and controlling polymorph formation by illuminating a material with non-absorbed polarized light as the material is thermally driven through a phase transition temperature. The invention also relates to a method of assessing polymorph formation by Raman spectroscopy and/or imaging of the material as it is thermally driven through a phase transition temperature.

The methods described herein can be used to make two- or three-dimensional Raman chemical images of a material as it is thermally driven through a phase transition temperature.

In one embodiment, a linearly polarized laser beam of a non-absorbing wavelength is directed onto a material held in a temperature-controlled thermal stage. Raman scattered light produced by the interaction of the material with the laser is collected as the material is thermally driven through a phase transition to higher temperatures and subsequently allowed to cool back through the phase transition to form a polymorph other than the original crystal form. The non-absorbed laser light both induces formation of a particular crystal form (polymorph) of the material and produces the Raman light or images by which the polymorphic form is controlled and verified.

The methods described herein are useful in a wide variety of applications, such as pharmaceutical manufacturing, optical data storage and security marking. Optical data storage refers to use of a medium of a material having a first polymorphic form to record data by altering “spots” on the medium (using the polymorph-induction methods described herein) such that selected spots are converted to a second, distinguishable polymorphic form, thereby “writing” the data on the medium. The polymorph detection methods described herein (or other methods, such as observing the reflectance of the selected polymorphs) can be used to detect the altered spots, thereby “reading” the data from the medium. The number of data states that can be represented at a single spot depends on the number of selectable and detectable polymorphs that can be generated at the spot (e.g., a dimorphic compound can indicate two data states, while a trimorphic compound can indicate three data states). Security marking refers to using the polymorph detection methods described herein to detect a selected polymorph of a compound that is intentionally included on or in a substance (e.g., in a drug tablet or on the surface of a ‘security tape’ label) and also refers to altering portions of a medium having a first polymorphic forms (using the polymorph-induction methods described herein) to create a “signature” of information to indicate the origin of the medium.

In another aspect, the invention relates to a method of encoding information by thermally assisted selection and laser writing of specific crystalline forms of materials.

### BRIEF SUMMARY OF THE SEVERAL VIEWS OF THE DRAWINGS

FIG. 1 depicts a schematic of a system for control and monitoring of polymorph formation.

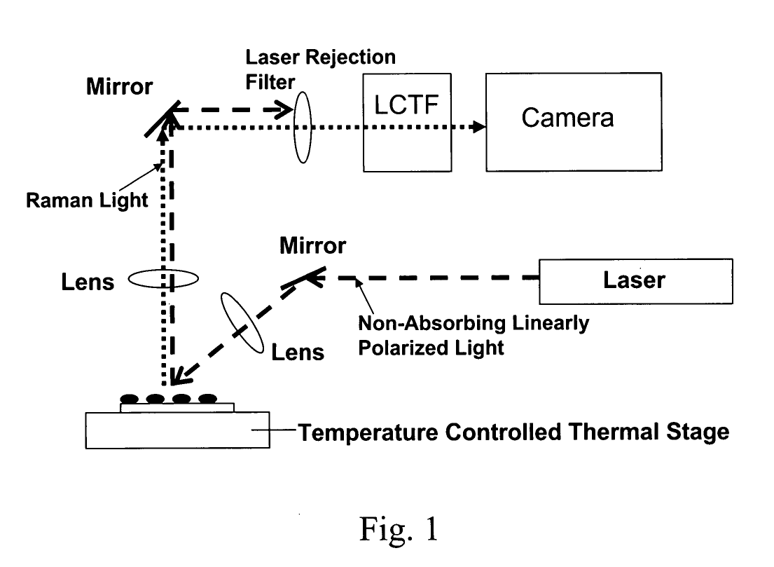

FIG. 2 is a pair of Raman spectra for forms I (solid line) and II (dashed line) of nabumetone.

FIG. 3 is a pair of Raman spectra for forms I (solid line) and II (dashed line) of nabumetone.

FIG. 4 is a pair of Raman spectra for forms I (upper spectrum) and II (lower spectrum) of nabumetone.

### DETAILED DESCRIPTION OF THE INVENTION

The invention relates to a method of crystallizing (i.e., inducing nucleation and/or crystal growth of) a selected polymorph of a substance. These methods can be used to synthesize a particular crystalline form of the substance, or to create a crystalline form of the substance upon the surface of which a different polymorph crystallizes, for example. Coupled with the polymorph analysis methods described herein, these crystallization methods can be used to form polymorphs of a substance which, if not of the desired type, can be discarded, melted and re-formed, or otherwise handled. The polymorph crystallization methods involve irradiating a compound with non-resonant laser radiation as it is thermally driven through a phase transition temperature (e.g., the melting point of the compound). These controlled crystallization methods permit selective synthesis of one or more polymorphic forms of a substance.

In another aspect, the invention relates to methods of assessing the polymorphic form of a substance using a Raman spectroscopic property of the substance. The method is useful, for example, for assessing relative amounts of crystalline forms of particles in mixtures containing multiple polymorphs of the same substance. The methods can be used for a variety of other purposes, such as assessing the polymorphic form(s) of a compound resulting from varied preparative methods, observing conversion from one polymorph to another, analyzing multi-component particulate mixtures, and dynamically assessing crystal nucleation and growth.

Use of the Raman spectroscopic polymorphism assessment methods described herein have a number of advantages over prior methods of polymorphism analysis. For example, polymorphism information can be directly attributed to individual particles of a substance, or even specific regions of a solid particle. The methods do not require separation of polymorphic forms or spectral unmixing techniques in order to characterize mixtures of polymorphs. Furthermore, the same methods can be used to characterize the geometric properties, such as particle size distribution (PSD), of drug and other particles in mixtures such as inhalable pharmaceutical products. Thus, the methods described herein are capable of more fully characterizing particulate materials (i.e., by geometric, chemical, and polymorphic analysis of individual particles in a mixture, or even by analysis of multiple intra-particle regions).

Briefly summarized, the methods comprise irradiating a substance, assessing Raman-shifted radiation scattered from the substance, and correlating that scattered radiation with the polymorphic form of the substance. The Raman spectroscopic information can be further used to identify (or confirm the identity of) the chemical identity of the substance of interest, a geometric property of a particle of the substance, other substances in the field of view, or some combination of these.

Definitions

As used herein, each of the following terms has the meaning associated with it in this section.

A “polymorph” is a single, homogenous crystalline form of a chemical compound that is capable of crystallizing in a plurality of distinct crystalline forms in which the crystal structures differ with respect to the orientation and position of the compound molecules forming the crystal lattice. Polymorphs include solventomorphs, which are crystalline solids in which solvent molecules are incorporated into the crystal lattice at specific and periodic locations that vary among different solventomorphs.

A “particle of a substance” is an entity having a phase boundary with one or more surrounding entities, wherein the entity comprises the substance. Examples of a particle of a substance include a solid phase of the substance surrounded by a liquid or gaseous phase, a first liquid phase that comprises the substance and is surrounded by a second liquid phase that substantially does not comprise the substance, and a first solid phase of the substance in one polymorphic form surrounded by a second solid phase of the substance in a second polymorphic form. A particle can consist entirely or essentially of the substance, or the particle can comprise other materials.

A particle is “effectively immobilized” if it is maintained in a location and an orientation that do not substantially change during the period of Raman scattering analysis described herein.

“Bandwidth” means the range of wavelengths in a beam of radiation, as assessed using the full width at half maximum method.

“Bandpass” of a detector or other system means the range of wavelengths that the detector or system can distinguish, as assessed using the full width at half maximum intensity method.

The “fill width at half maximum” (“FWHM”) method is a way of characterizing radiation including a range of wavelengths by identifying the range of contiguous wavelengths that over which the magnitude of a property (e.g., intensity or detection capacity) is equal to at least half the maximum magnitude of that property in the radiation at a single wavelength.

“Spectral resolution” means the ability of a radiation detection system to resolve two spectral peaks.

A compound is irradiated with “non-resonant” laser radiation if it is irradiated with laser radiation that is not absorbed by the compound.

Detailed Description

Polymorph Assessment

The invention relates to methods of assessing the polymorphic form of a substance using a Raman spectroscopic property of the substance. The methods comprise irradiating a substance, assessing Raman-shifted radiation scattered from the substance, and correlating that scattered radiation with the polymorphic form of the substance.

In an important embodiment, the substance is a component of a particulate solid preparation which can contain one or more other solid particulate ingredients (or liquid ingredients) in addition to the substance. For instance, the substance can be one particulate ingredient of a pharmaceutical preparation intended to be dispersed and inhaled. Alternatively, the substance can be a solid particle(s) suspended in a fluid or a particulate compressed with other solid ingredients in a solid tablet form. The precise form of the substance is not critical to operation of the method, at least insofar as the substance can be irradiated and Raman-shifted radiation scattered from the substance can be detected. In this embodiment, the polymorphic form of a component of a pharmaceutical preparation can be assessed, for example for quality control purposes or for assessment of storage stability.

The methods are suited for assessment of the polymorphic form of microscopic particles or regions of a substance, such as solid drug particles in a powdered, aerosol, or compressed tablet composition of a pharmaceutical preparation. When the polymorphic form of a substance in a microscopic field is assessed, the entire microscopic field can be irradiated at once or smaller portions of the field can be illuminated. Alternatively, a relatively small portion of the microscopic field can be irradiated while Raman-shifted light scattered from that portion of the field is assessed, after which a different portion of the field can be irradiated and assessed.

While the substance (e.g., a portion of a microscopic field containing the substance) is being irradiated, Raman-shifted radiation scattered by the illuminated substance is assessed. The characteristics of the Raman-shifted scattered radiation depend on the chemical identity of the irradiated substance, including the polymorphic form of the substance. By assessing the Raman characteristics of light scattered from the illuminated portion of a sample, the chemical identity and polymorphic form(s) of the substance(s) present in the illuminated portion can be determined.

The Raman spectral characteristics of polymorphs of a substance can be distinguished. Raman spectra of homogenous known polymorphs can be collected and archived, such as in an electronic memory storage system (e.g., a computer) or as a printed spectrum. An unknown polymorphic form of the same compound can be identified by comparison of its Raman characteristics with archived spectra. A previously unknown polymorph can be identified by the dissimilarity of its Raman spectral characteristics and those of known polymorphs. Even in the absence of archived Raman spectral data, polymorphs can be distinguished from one another by their Raman spectral differences. Thus, different Raman spectra obtained for two crystals of the same compound can be correlated as arising from different polymorphs of the compound, even if neither polymorph has been further characterized. The different packing arrangements of a compound in two different polymorphs affects the interactions between molecules of the compound in the polymorphs. The differences in the intermolecular interactions between two polymorphs can perturb the energy states of the intramolecular bonds in the compound. Perturbations in the energy states of the bonds is manifested as differences in the Raman spectra of the polymorphs.

Raman Spectroscopy

Raman spectroscopy provides information about the vibrational state of molecules. Many molecules have atomic bonds capable of existing in a number of vibrational states. Such molecules are able to absorb incident radiation that matches a transition between two of its allowed vibrational states and to subsequently emit the radiation. Most often, absorbed radiation is re-radiated at the same wavelength, a process designated Rayleigh or elastic scattering. In some instances, the re-radiated radiation can contain slightly more or slightly less energy than the absorbed radiation (depending on the allowable vibrational states and the initial and final vibrational states of the molecule). The result of the energy difference between the incident and re-radiated radiation is manifested as a shift in the wavelength between the incident and re-radiated radiation, and the degree of difference is designated the Raman shift (RS), measured in units of wavenumber (related to inverse wavelength). If the incident light is substantially monochromatic (single wavelength) as it is when using a laser source, the scattered light which differs in frequency can be more easily distinguished from the Rayleigh scattered light.

Because Raman spectroscopy is based on irradiation of a sample and detection of scattered radiation, it can be employed non-invasively and non-destructively to analyze biological samples in situ. Thus, little or no sample preparation is required. In addition, water exhibits very little Raman scattering, and Raman spectroscopy techniques can be readily performed in aqueous environments.

The Raman spectrum of a material can reveal the molecular composition of the material, including the specific fimctional groups present in organic and inorganic molecules. Raman spectroscopy is useful for detection of pharmaceutical and other chemical agents because most, if not all, of these agents exhibit characteristic ‘fingerprint’ Raman spectra, subject to various selection rules, by which the agent can be identified. Raman peak position, peak shape, and adherence to selection rules can be used to determine molecular identity and to determine conformational information (e.g., crystalline phase, degree of order, strain, grain size) for solid materials. The Raman spectrum of a solid material is also dependent on the crystalline form (or lack of crystalline form) in which the material occurs. Thus, polymorphs of a polymorphic substance can be distinguished by the Raman characteristics of light scattered by the polymorphs, such as differences in the shape of their Raman spectra, the occurrence (or non-occurrence) of a peak at a characteristic Raman shift value, the ratio of Raman peaks, the intensity at a single Raman shift value, or some combination of these.

In the past several years, a number of key technologies have been introduced into wide use that have enabled scientists to largely overcome the problems inherent to Raman spectroscopy. These technologies include high efficiency solid-state lasers, efficient laser rejection filters, and silicon CCD detectors. In general, the wavelength and bandwidth of light used to illuminate the sample is not critical, so long as the other optical elements of the system operate in the same spectral range as the light source.

In order to detect Raman scattered light and to accurately determine the Raman shift of that light, the sample should be irradiated with substantially monochromatic light, such as light having a bandwidth not greater than about 1.3 nanometers, and preferably not greater than 1.0, 0.50, or 0.25 nanometer. Suitable sources include various lasers and polychromatic light source-monochromator combinations. It is recognized that the bandwidth of the irradiating light, the resolution of the wavelength resolving element(s), and the spectral range of the detector determine how well a spectral feature can be observed, detected, or distinguished from other spectral features. The combined properties of these elements (i.e., the light source, the filter, grating, or other mechanism used to distinguish Raman scattered light by wavelength) define the spectral resolution of the Raman signal detection system. The known relationships of these elements enable the skilled artisan to select appropriate components in readily calculable ways. Limitations in spectral resolution of the system (e.g., limitations relating to the bandwidth of irradiating light) can limit the ability to resolve, detect, or distinguish spectral features. The skilled artisan understands that and how the separation and shape of Raman scattering signals can determine the acceptable limits of spectral resolution for the system for any of the Raman spectral features described herein.

A single set of optics can be used both for irradiation of a sample and detection of Raman-shifted scattered radiation, since the Raman-shifted radiation differs in wavelength from the illuminating radiation. Some samples will exhibit fluorescence at wavelengths that overlap the wavelengths of Raman-shifted scattered radiation. However, if such fluorescence occurs at a wavelength of interest for assessing Raman-shifted radiation in a sample, then a subtractive method can be used to correct for such fluorescence and prevent fluorescent emissions from obscuring relevant scattering data. In such a method, the intensity of (fluorescently emitted and/or Raman scattered) light emitted from a first portion of the sample at which the substance is known or believed to be absent is assessed and subtracted from the intensity of (fluorescently emitted and/or Raman scattered) light emitted from a second portion of the sample at which the substance is present. Because the intensity of background fluorescent emissions should normally be indistinguishable, the difference in intensity between the first and second portions will represent the intensity of Raman scattered light from the substance at the second portion. Of course, other known mathematical strategies can be used to distinguish broadband fluorescent emissions from narrower Raman radiation. As described herein, that Raman scattering data can be used to assess the polymorphic form of the substance at the second portion of the sample.

In one embodiment, a substance for which Raman-shifted scattered radiation is to be assessed in order to determine the polymorphic form of the substance is irradiated using a source through an optical path that is distinct from the optical path through which Raman-shifted scattered radiation is assessed. In such a dark-field arrangement, there is a lesser likelihood that illuminating radiation will interfere with detection optics. Such an arrangement permits use of lower-power illumination than might otherwise be required, permitting use of compact solid-state laser sources, for example.

Raman Chemical Imaging

Spectroscopic methods can be extended to chemical imaging (also known as spectroscopic imaging) techniques through the use of imaging spectrometers such as liquid crystal imaging spectrometers. The development of this technology in recent years has enabled the practice of widefield spectroscopic imaging to develop and mature. Chemical imaging is a versatile technique that is well suited to the analysis of complex heterogeneous materials such as drug particles in a complex mixture of particulate solids. Chemical imaging provides a potential solution for obtaining both qualitative and quantitative image information about molecular composition and morphology materials allowing a more accurate and more rapid analysis than traditional imaging or ‘wet’ chemical methods.

Raman chemical imaging (RCI) combines Raman spectroscopy with digital imaging for molecule-specific and polymorph-specific analysis of materials. This technology allows images of samples to be constructed by recording Raman scattered light at discrete wavelengths emanating from selected locations in an illuminated sample. A spectrum is generated corresponding to millions of spatial locations at the sample surface by tuning the liquid crystal imaging Raman spectrometer over a range of wavelengths and collecting images intermittently. Depending on the materials, depth-related information can also be obtained by using different excitation wavelengths or by capturing Raman chemical images at incremental planes of focus. Contrast is generated in the images based on the relative amounts of Raman scatter that is generated by the different species located throughout the sample. Since a Raman spectrum is generated for each pixel location, univariate and/or multivariate (i.e., chemometric) analysis tools such as correlation analysis, Principal Component Analysis (PCA) and factor rotation, including Multivariate Curve Resolution (MCR) can be applied to the image data to extract pertinent information. If necessary, spectral unmixing methods, such as those described in the co-pending U.S. patent application 10/812,233, filed 29 Mar. 2004, can be used to extract chemical identity and/or polymorph information from a pixel at which a plurality of chemicals and/or polymorphs occur.

Preferably, Raman scatter from a sample of a substance is assessed at a plurality portions of a microscopic field (e.g., multiple particles of a powdered solid composition or at multiple locations on the surface of a compressed tablet). Depending on the resolution of the Raman scatter detector, the method can be used to assess the polymorphic form of the substance in every particle of a powder in a microscopic field (or a significant portion thereof) or to prepare an image that maps the location of polymorphs of a substance on the surface (or in a layer) of a tabletted form of a pharmaceutical composition, for example. By assessing Raman scatter at adjacent or overlapping areas in a single microscopic field, a two-dimensional image of the polymorphs present in the field can be generated. By adjusting the focal plane perpendicularly to the plane in a suitable substrate (i.e., one in which illuminating radiation can penetrate to the focal plane and Raman scattered light can travel from the focal plane to the detector), a three-dimensional image of the polymorphs in the microscopic field can be made.

A spatial resolving power of approximately 250 nanometers has been demonstrated for Raman chemical imaging using laser illumination at visible wavelengths. This is almost two orders of magnitude better than infrared imaging that is typically limited to 20 microns due to diffraction. The theoretical limit of the resolving power of an RCI instrument corresponds to the Abbe diffraction limit. In addition, image definition (based on the total number of imaging pixels) can be very high for RCI based on liquid crystal optics because high pixel density detectors (often 1 million plus detector elements per field of view) can be used, so that spatial resolution of the RCI image is limited by the spatial resolution of the image capture optics, rather than by the resolution of the sensor pixels. Thus, Raman chemical imaging methods are useful for charactering the polymorphic form(s) of crystals occurring in substantially any pharmaceutical preparation and in many other solid and particulate compositions.

An apparatus for Raman chemical imaging has been described by Treado in U.S. Pat. No. 6,002,476, and in U.S. patent application 09/619,371, filed 19 Jul. 2000, which are incorporated herein by reference. Other descriptions of Raman chemical imaging are U.S. patent application 09/800,953, filed 7 Mar. 2001; U.S. patent application 09/976,391, filed 21 Oct. 2001; U.S. patent application 10/185,090, filed 27 Jun. 2002; U.S. patent application 10/184,580 filed 27 Jun. 2002; U.S. provisional patent application 60/144,518, filed 19 Jul. 1999; U.S. provisional patent application 60/347,806, filed 10 Jan. 2002; U.S. provisional patent application 60/144,518, filed 19 Jul. 1999; U.S. provisional patent application 60/187,560, filed 28 Mar. 2000; U.S. provisional patent application 60/239,969, filed 13 Nov. 2000; U.S. provisional patent application 60/301,708 filed, 28 Jun. 2001; and U.S. provisional patent application 60/422,604, filed 21 Nov. 2002. Each of the foregoing patents and applications is incorporated herein by reference.

RCI instrument configurations can include platforms based on an RCI microscope, for example. An example of a commercially available device which is suitable for use in the methods described herein is a laboratory or transportable field Raman microscope such as the FALCON Raman microscope (TM; ChemImage Corporation, Pittsburgh, Pa.).

In order to ensure proper peak positions in dispersive Raman and RCI data, the RCI instrument should be calibrated using a NIST-accepted calibration standard for Raman spectrometers. A common standard is acetaminophen. If the identity(ies) of components of the sample other than the substance of interest are known, then Raman spectral data for each of those components can be generated. This information permits identification of appropriate portions of the Raman spectrum to scan during RCI data acquisition to avoid overlapping Raman scattering peaks. If one or more standard preparations of the substance of interest (e.g., each standard preparation including a single polymorph of the substance) are available, then those standard preparations can also be used to confirm calibration of the Raman spectroscopy system and to select Raman characteristics by which the polymorphs can be distinguished.

Typically, a Raman peak that both is distinctive of a polymorph of the substance of interest and exhibits an acceptable signal-to-noise ratio will be selected. Multiple Raman shift values characteristic of the polymorph(s) can be assessed. If the sample includes unknown components, then the entire Raman spectrum can be scanned during RCI data acquisition, so that the contributions of any contaminants to the data can be assessed.

RCI Microscope-Based System

An RCI microscope such as the FALCON (TM) system described above combines in a single platform a solid state laser for sample excitation, a refractive optical microscope base, which is equipped with infinity-corrected microscope objectives, an automated XYZ translational microscope stage and a quartz tungsten halogen (QTH) lamp and/or a mercury (Hg) lamp. Also a part of the microscope system is an analog color charge-coupled device (CCD) detector for ordinary optical image collection and digital image collection, a liquid crystal imaging spectrometer for spectroscopic image wavelength selection, a thermoelectrically (TE) cooled silicon (Si) CCD detector for Raman chemical image capture, and a remote, dispersive monochromator equipped with a CCD detector for dispersive spectral collection.

Ordinary optical imagery of the sample can be obtained using a mirror, beamsplitter, or prism arrangement inserted into the turret wheel of the microscope by collecting an image with an analog or digital color or monochrome CCD or CMOS detector. In spectroscopic imaging mode, the spectroscopic image is coupled through a liquid crystal imaging spectrometer and collected on a Si CCD detector for RCI. A central processing unit, typically a PENTIM(RTM) computer, is used for spectroscopic image collection and processing. The analog color CCD, Si CCD, automated XYZ translational microscope stage (controlled by way of a controller), and liquid crystal imaging spectrometer (controlled by way of a liquid crystal imaging spectrometer controller) can be operated with commercial software, such as CHEMAQUIRE (TM; Chemimage Corporation, Pittsburgh, Pa.) in conjunction with CHEMANALYZE (TM; ChemImage Corporation, Pittsburgh, Pa.).

By introducing a polarization sensitive beam splitting element in the optical path prior to the liquid crystal imaging spectrometer, a portion of the signal from the sample may be coupled to a remote dispersive spectrometer. This allows conventional spectroscopic tools to be used to gather spectra for traditional, high-speed spectral analysis. The spectrometers can be any of a fixed filter spectrometer, a grating-based spectrometer, a Fourier transform spectrometer, and an acousto-optic spectrometer, for example.

Preferably, liquid crystal (LC) imaging spectrometer technology is used for wavelength selection. The LC imaging spectrometer can, for example, be one of a Lyot liquid crystal tunable filter (LCTF), an Evans Split-Element LCTF, a Solc LCTF, a ferroelectric LCTF, a liquid crystal Fabry Perot (LCFP), a hybrid filter that combines two or more of the above-mentioned LC filter types, and one of the above mentioned filter types in combination with fixed bandpass and bandreject filters, which can be of the dielectric, rugate, holographic, color absorption, acousto-optic or polarization filter types.

The RCI microscope can be used as a volumetric imaging instrument by moving the sample through focus in the Z-axial dimension, collecting images in and out of focus and reconstructing a volumetric image of the sample in software. For samples having some volume (bulk materials, surfaces, interfaces, interphases), volumetric chemical imaging has been shown to be useful for failure analysis, product development, and routine quality monitoring. The potential also exists for performing quantitative analysis simultaneously with volumetric analysis. Volumetric imaging can be performed in a non-contact mode without modifying the sample through the use of numerical confocal techniques, which require that the sample be imaged at discrete focal planes. The resulting images are processed and reconstructed and visualized. Computational optical sectioning reconstruction techniques based on a variety of strategies have been demonstrated, including nearest neighbors and iterative deconvolution.

Non-Raman Spectroscopic Analysis

One or more of a variety of spectroscopic methods other than Raman spectroscopy can be performed in parallel with the Raman spectroscopic analysis described herein. In many instances, the other spectroscopic method can be performed using many of the same optical components (e.g., lenses and mirrors) used for Raman analysis. Examples of suitable non-Raman spectroscopic analytical methods include visible light microscopy, polarized light microscopy, bright-field crossed-polarization microscopy, birefringence imaging, and absorbance spectrometry. By way of example, an optical microscopic image of a powder can be generated simultaneously with Raman spectroscopic analysis of the field of view. The images and/or information generated by the Raman and other spectroscopic methods can be combined optically or electronically (e.g., by display of overlapping data on a computer monitor).

In one embodiment, a spectroscopic method that can be performed relatively rapidly and/or with relatively little computing power is used to identify areas of or entities within a microscopic field for which Raman scattering analysis should be performed (or, conversely, need not be performed). In this way, collection of Raman scattering data can be limited to areas of a microscopic field at which the substance of interest occurs. Examples of suitable non-Raman spectroscopic methods include absorbance, fluorescence, diffraction, polarization, and microscopic methods. In one embodiment, the microscopic field is illuminated with radiation that can be used in more than one optical and/or spectroscopic method (e.g., incident laser light useful for Raman scattering, fluorescence spectroscopy, and optical microscopy). If the substance of interest exhibits a characteristic Raman scattering that can be assessed at a single Raman shift value, then Raman scattering analysis at that Raman shift value alone can rapidly identify areas of the microscopic field that are appropriate for more extensive Raman scattering analysis (i.e., areas of the microscopic field that contain, or appear to contain, the substance of interest).

Microscopic methods can be used for assessing the morphology of entities in a sample. Suitable microscopic methods include scanning electron microscopy, differential interference contrast microscopy, brightfield reflectance microscopy, polarized light microscopy, and fluorescence microscopy.

Absorption and fluorescence measurements based upon UV-visible or NIR irradiation allow qualitative and quantitative determination of inorganic and organic species in a microscopic field. To the extent that specific absorbance or fluorescence information about a substance of interest is known, analysis of radiation absorbed or fluoresced (i.e., fluorescently emitted) by a sample can be informative regarding the occurrence of that target in the sample. Absorption and fluorescent spectroscopic methods are therefore useful for identifying entities in a sample for which Raman scattering analysis need not be performed or should be performed in order to assess the polymorphic form(s) of a substance of interest.

Spectroscopic methods can be extended to imaging techniques through the use of imaging spectrometers such as liquid crystal imaging spectrometers. The development of this technology in recent years has enabled the practice of spectroscopic imaging to develop and mature. Spectroscopic imaging is a versatile technique that is well suited to the analysis of complex heterogeneous materials. Spectroscopic imaging provides a potential solution for obtaining both qualitative and quantitative image information about molecular composition and morphology of crystalline particles or regions of a substance in a sample. Non-Raman spectroscopic imaging methods can also help to identify portions of a sample in which a substance of interest occurs in an amorphous form.

Particle Analysis Methods

The suitability of the Raman spectroscopic methods described herein for analysis on a sub-micron scale and their compatibility with other spectroscopic methods allow those methods to be employed together with traditional particle analysis methods. Acquired RCI and/or other spectroscopic (e.g., optical) images can be subjected to one or more univariate and/or multivariate image processing strategies to extract geometric information about particles or regions of the compound(s) and polymorph(s) that can be identified from the Raman data.

Many image processing strategies are described in the art, and selection of one or more such strategies is within the level of ordinary skill in this field. Various software packages are also commercially available which are able to translate two- and three-dimensional RCI data sets to geometric properties for particles (i.e., programs that calculate geometric properties of solids from RCI data). An example of suitable software for use with the FALCON (TM) RCI microscope system is the CHEMANALYZE (TM) software package available from ChemImage Corporation (Pittsburgh, PA).

A useful method for creating an easily-manipulated image which can be used for geometric property determination is creation from RCI data (or other spectroscopic information, such as an optical image) of one or more binary image frames, each corresponding to a particular Raman shift value and/or a particular plane of focus. For example, software can be used to assign a value of “1” to pixels that contain spatial/spectral information characteristic of the substance of interest and a value of “0” to pixels containing spatial/spectral information not characteristic of the substance. Once Raman images have been binarized, appropriate particle sizing software is applied to the processed data to determine molecule-specific particle sizes.

Typical geometrical parameters that are used to describe particle size based on two-dimensional data include the following: area (cross-sectional area of particle); perimeter (boundary length of particle); Feret diameter 1 (horizontal distance across particle); Feret diameter 2 (vertical distance across particle - i.e., Feret diameter along axis perpendicular to Feret diameter 1); maximum chord length (maximum distance across particle); shape factor (i.e., the value of the formula (4×pi×Area)/Perimeter2); aspect ratio (Feret diameter 1/Feret diameter 2).

Typical geometrical parameters that are used to describe particle size based on three-dimensional data include the following: volume (volume of the particle); surface area (surface of the particle); Feret diameters (three, orthogonal to one another); maximum chord length (maximum distance across particle); various shape factors, and various measures of aspect ratios or sphericity of the particle.

These geometrical parameters can be determined using the methods described herein or calculated from geometrical parameters that can be determined using such methods.

The dimensional limits of the particle analysis methods described herein are defined by the RCI or other spectroscopic imaging system being used. Currently, the minimum spatial differentiation limit of the Chemimage FALCON (TM) RCI microscope is believed to be about 200-250 nanometers, meaning that geometrical properties of particles smaller than this could not be effectively assessed using that system. However, there is no effective theoretical lower limit to the size of particles that can be assessed using these methods, and the methods described herein can be readily applied to any new instrument having a lower spatial differentiation limit than the FALCON (TM) device.

Combining the geometric properties thus calculated with chemical and polymorph identity information generated by Raman spectroscopic analysis permits greater characterization of complex powdered and solid mixtures than was previously possible. By way of example, a powdered pharmaceutical composition can be analyzed using Raman and non-Raman spectroscopic information to yield information about the size and composition of particles in the composition as well as information about the polymorphic form(s) of a substance in those particles. Using the methods described herein, two- and three-dimensional images of the spatial distribution of the particles, components, and polymorphs can be generated.

Calorimetry

Crystalline forms of a substance can melt or interconvert at temperatures characteristic of the polymorphic form of the substance. The amount of heat necessary to achieve these transformations is also characteristic of the polymorph. Calorimetric methods (e.g., differential scanning calorimetry) can be used to observe phase and polymorphic transitions that solid substances undergo. However, such methods record energy flux as a function of temperature change and are unable, by themselves, to provide information about the phases and/or polymorphic forms that occur during the period of analysis.

By coupling Raman spectroscopy with a calorimetric method, one can determine the crystalline and polymorphic form of the substance throughout the period of calorimetric analysis. Thus, coupled Raman and calorimetric methods can be used to understand inter-polymorphic transformations of materials better than either method alone and better than is presently possible. Using the Raman spectroscopic methods described herein in conjunction with a calorimetric analytical method, it is possible to perform calorimetric analysis of mixtures of polymorphs of a substance, because energy fluxes can be correlated with phase and/or polymorphic form changes in the substance. These coupled methods can also be used to understand the calorimetric behavior of a substance in a mixture of the substance of interest and other compounds. Energy fluxes of the mixture can be correlated with phase and/or polymorphic form changes in the substance or in another compound in the mixture and the relative contributions of the components of the mixture can be assessed.

Sample Immobilization

In some embodiments, such as analysis of powders, the present invention requires effective immobilization of the substance of interest on a substrate having properties conducive to Raman chemical imaging. Such a substrate should preferably be flat, resistant to damage or modification upon laser illumination, resistant to thermal expansion, relatively Raman inactive (i.e., does not exhibit Raman scattering of the radiation with which it is illuminated), and non-interferent with the Raman light scattered from the sample. Analysis of larger or more structurally rigid samples (e.g., a tablet or a layer of powder that obscures the substrate upon which it rests) can be performed with little regard for the substrate, since little or none of the light used to irradiate the sample will be scattered by the substrate.

An appropriate choice of substrate is an aluminum-coated glass microscope slide. Ordinary glass microscope slides can also be used, at least with certain laser illumination wavelengths that are apparent to skilled artisans and/or readily empirically determined. Powdered or aerosolized particles or particles suspended in a liquid can be applied to surface of such a slide and any liquid in the composition can be allowed to dry. Alternatively, compositions in which particles of the substance of interest are suspended in a fluid can be frozen on the surface of a slide (e.g., by cooling the slide and spraying an aerosolized particle suspension thereon). As another alternative, a composition comprising particles of interest can be suspended in a polymer resin that is thereafter cured to immobilize the particles. The resin can be cured in place or sliced after curing.

If a liquid preparation of particles (e.g., solid particles suspended in liquid or a particulate (i.e., dispersed) liquid phase suspended in a continuous liquid phase) is to be analyzed, the preparation can be immobilized by maintaining a thin layer of the liquid under conditions (e.g., high humidity for aqueous preparations) in which the liquid will not evaporate. Alternatively, liquid samples can be sandwiched between transparent glass or plastic slides, optionally having a spacer interposed between the slides to yield a liquid layer of defined thickness.

In order to collect RCI data, substantially immobilized particles are brought into focus under the microscope and the appropriate data collection parameters for the instrument are set. Raman chemical image(s) are collected. Brightfield and other supporting optical imagery can also be acquired at this time to provide complimentary spatial/birefringence information in addition to the RCI data.

Multiple Particle Analyses

If the sample being analyzed comprises particles or regions having distinguishable Raman spectral properties, then the particle analytical methods described herein can be used to assess polymorphic and geometric properties of all of the spectrally-distinguishable types of particles in the composition. By way of example, if an aerosolized pharmaceutical composition contains two polymorphic forms of an active agent that can be differentiated by their Raman scattering peaks, then RCI data can be collected at two Raman shift values - one Raman shift value characteristic of one polymorphic form, and another Raman shift value characteristic of the other polymorphic form. This RCI data set will include information sufficient to describe geometric properties of both polymorphic forms.

Other spectral properties can be used to describe geometric properties of particles that cannot be identified by a characteristic Raman shift value. By way of example, if the cornposition described in the previous paragraph also contains a third particle type which can be differentiated from all other components of the composition by a characteristic fluorescent peak, then geometric properties of that third particle type can be assessed by analysis of fluorescent imaging data obtained for the characteristically-fluorescing particle.

If all particle types except one in a composition can be characterized by a Raman shift value or other spectroscopic characteristic, then all particles that do not exhibit that Raman shift and/or other spectroscopic characteristic can be presumed to be the remaining particle type, and optical microscopy data can be used in combination with RCI and other spectroscopic imaging data to assess one or more geometric properties of the remaining particle type.

Induction of Crystallization by Non-Resonant Laser Illumination

An important aspect of the invention is that a selected polymorph of a compound can be crystallized by irradiating a solid form (i.e., crystalline or amorphous) of the compound with non-resonant laser radiation as the compound is thermally driven through a phase transition temperature. This treatment induces nucleation of crystals of the compound, and the polymorphic form of those crystals depends on the polymorphic form of the crystallization nuclei.

It is known that seeding a supersaturated solution of a compound with a particular polymorph of that compound will often result in crystallization of that polymorph in the solution. However, it is also known that seeding a supersaturated solution of a compound with a first polymorph of that compound will sometimes induce crystallization of a second polymorph, presumably because the second polymorph crystallizes on the surface of the first.

Thus, in order to induce crystallization of a selected polymorph from a saturated solution, it is useful to know whether crystallization of the selected polymorph is induced by seeding the supersaturated solution with that polymorph, by a different polymorph, or both. The methods described herein offer an alternative to polymorph selection via precipitation from a supersaturated solution. Once an appropriate polymorphic form of crystallization nuclei is determined for a compound, non-resonant laser illumination conditions can be selected such that crystallization of the desired polymorph is induced upon thermally driving the compound through a phase transition temperature.

The identity of the phase transition through which the compound is thermally driven is not critical. By way of example, this process can include thermally driving a compound from below the melting point of a pre-existing polymorphic form to above its melting point (i.e., melting the compound) and thereafter thermally driving the temperature of the compound below the melting point of the same or a second polymorphic form of the compound. As another example, the thermal driving can include increasing the temperature of the compound from below the melting point of the compound to above a melting point, at which temperature a liquid crystalline form of a desired polymorphic form of the compound forms. A further example includes thermally driving a liquid (or gaseous) form of a compound below its melting (or sublimation) temperature to form a solid polymorphic form of the compound. Still further by way of example, the compound can be thermally driven from a temperature that favors a first solid polymorphic form of the compound to a temperature that favors a second solid polymorphic form (i.e., with no intervening solid-to-liquid or solid-to-vapor phase transitions). These phase transition temperatures are known for many compounds and are readily determinable for other compounds.

In many instances, it is not currently possible to predict a priori the non-resonant laser illumination conditions that will result in formation of crystallization nuclei of a desired polymorphic form. However, various illumination conditions can be investigated to correlate those conditions with the polymorph(s) formed, and such empirical observations (which can vary with the compound used) require no more than routine experimentation. The Raman spectroscopic methods described herein can be used to identify the polymorph(s) formed under any set of laser illumination conditions. Because the Raman scattering effect occurs essentially independent of the wavelength and other characteristics of the light used to illuminate a sample, the same irradiation can be used both to induce crystallization of a compound as it is thermally driven through a phase transition temperature and to assess the polymorphic form of crystals and crystallization nuclei formed. Alternatively, Raman analysis of crystals and nuclei can be performed using laser illumination of the solid compound under conditions known (or empirically determined) not to induce crystallization of the compound.

The characteristics of the radiation used for non-resonant laser illumination of a compound can influence the polymorph that is formed upon illumination and thermal phase translitionng of the compound For instance, altering one or more of the wavelength, intensity, duration, polarization (e.g., linear polarization), and pulsile nature (e.g., pulse periodicity and/or intensity) of the illuminating laser radiation can influence the polymorph that is obtained.

Coupling induction of crystallization by non-resonant laser illumination with Raman spectroscopic analysis of polymorphs allows the crystallization process to be followed in near real time. If Raman analysis of polymorph(s) in a discrete portion of the melt indicates that a crystallization nucleus having a non-desired polymorphic form has been generated in that portion, then the portion of the solution can be discarded or treated (e.g., by heating or dilution) in a way that reverses crystallization. In this way, controlled synthesis of crystallization nuclei can be achieved. Crystallization nuclei (or crystals) having a desired polymorphic form can thereafter be selected for combination with a larger quantity of the supersaturated solution for the purpose of inducing crystallization of a desired polymorph in the solution.

In order to induce crystallization of a desired polymorph, a compound can be irradiated with a single beam of a non-resonant laser or with multiple beams. The multiple beams can be derived from separate lasers or optically from a single laser.

Example

The invention is now described with reference to the following Example. This Example is provided for the purpose of illustration only, and the invention is not limited to this Example, but rather encompasses all variations which are evident as a result of the teaching provided herein.

Selective Crystallization of Nabumetone

The experiments in this example were performed to demonstrate that an energetically unstable polymorph of nabumetone (4-(6-methoxy-2-naphthalenyl)-2-butanone) could be reproducibly formed by non-resonant laser irradiation of solid nabumetone.

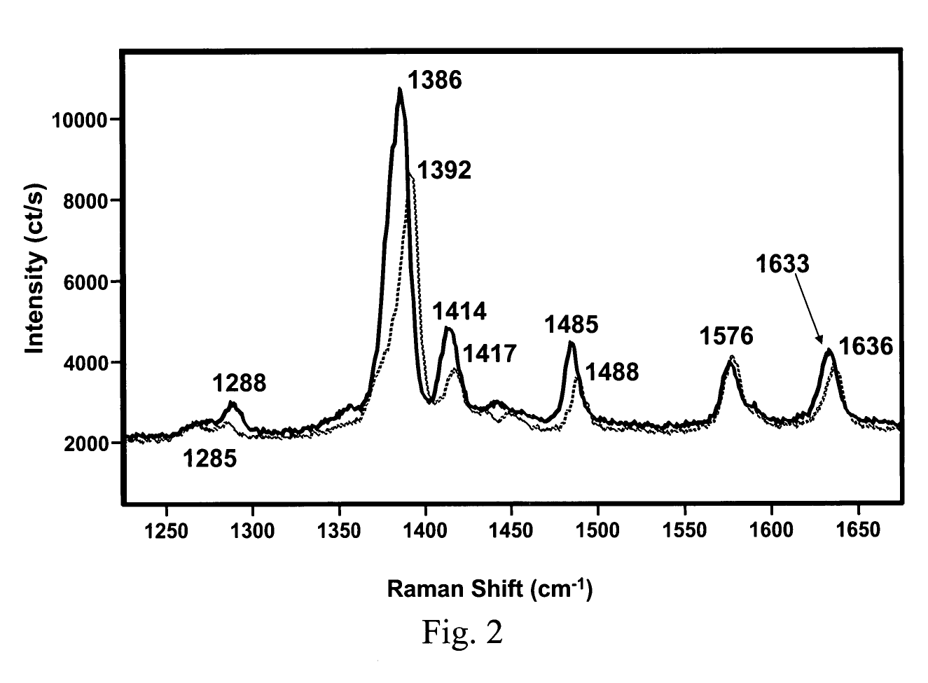

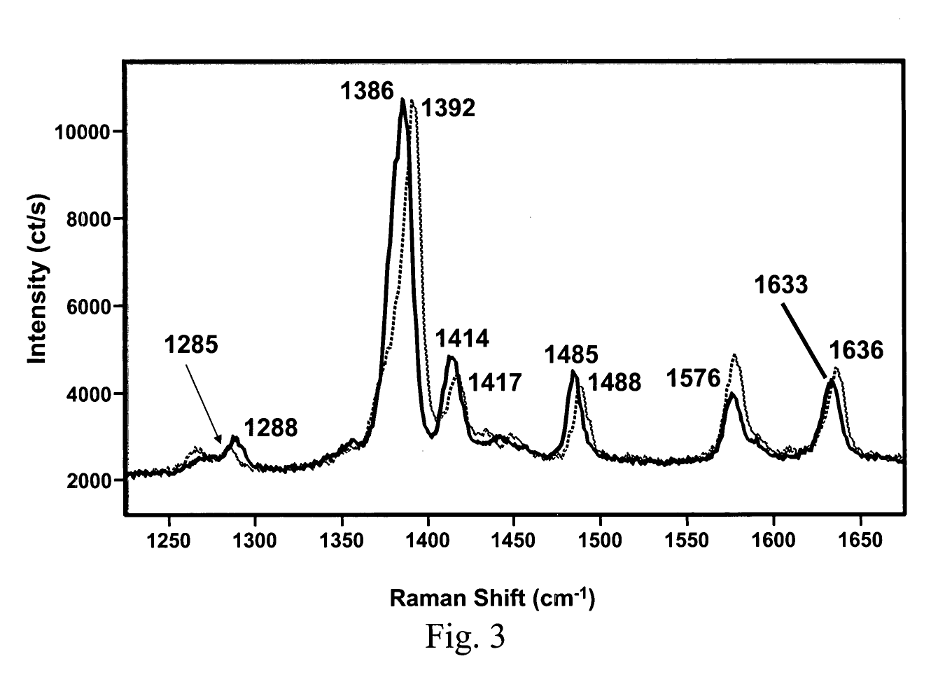

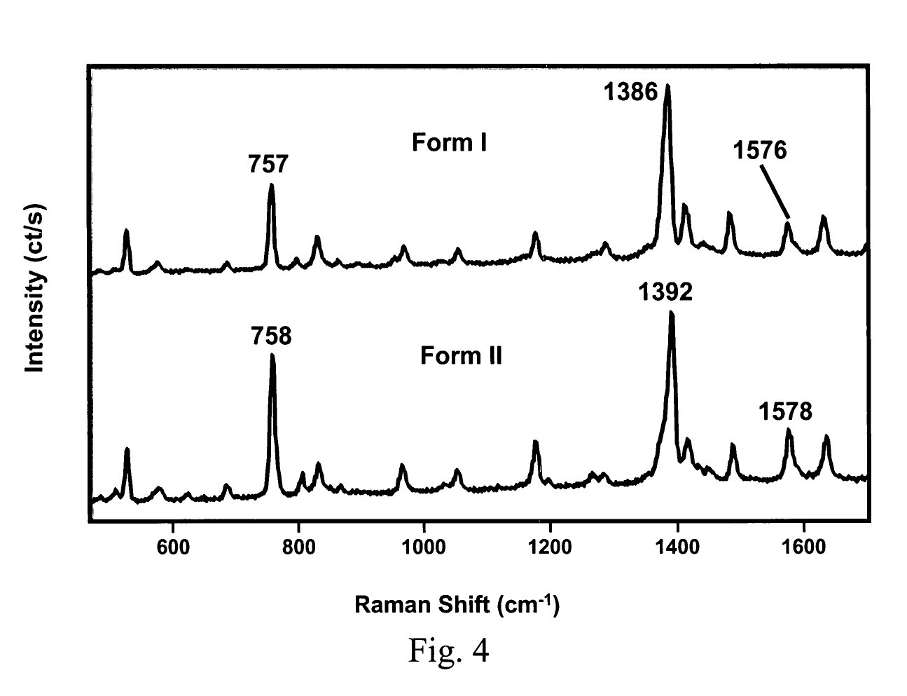

A solid crystalline form of nabumetone was illuminated with non-resonant laser radiation while the sample was thermally driven through a phase transition temperature. Subsequent Raman scattering analysis of the nabumetone demonstrated the presence of a second crystalline form of nabumetone in the laser-illuminated sample. The Raman spectra of the two polymorphs of nabumetone are shown in FIGS. 2, 3, and 4.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1. A method of generating a selected polymorph of a compound, the method comprising irradiating the compound using non-resonant radiation as the compound is thermally driven through a phase transition.

2. The method of claim 1, wherein a solid form of the compound is irradiated.

3. The method of claim 2, wherein the selected polymorph crystallizes as the compound is thermally driven through a liquid-to-solid phase transition.

4. The method of claim 1, wherein the compound is irradiated using the non-resonant radiation as the compound is thermally driven through a solid-to-liquid phase transition and thereafter through a liquid-to-solid phase transition.

5. The method of claim 1, wherein the radiation is provided using a laser.

6. The method of claim 1, wherein the compound is nabumetone.

7. The method of claim 1, comprising irradiating multiple portions of a substrate comprising the compound to yield a substrate having multiple portions comprising the selected polymorph.

8. The method of claim 7, wherein the substrate is an optical data storage medium.

9. The method of claim 7, wherein the substrate is a component of a security device.

10. The method of claim 7, wherein the substrate is a component of an identification device.

11. The method of claim 1, further comprising assessing Raman-shifted radiation scattered from the compound, and correlating the Raman-shifted radiation with the polymorphic form of the compound.

12. The method of claim 11, wherein the Raman-shifted radiation is the non-resonant laser radiation scattered from the compound.

13. The method of claim 1 1, further comprising ablating the compound if the polymorphic form of the compound is not the selected polymorph.

14. The method of claim 11, further comprising repeating the thermal driving through the phase transition if the polymorphic form of the compound is not the selected polymorph.

15. The method of claim 14, further comprising altering at least one of the wavelength, intensity, duration, polarization, and pulsile nature of the laser irradiation during the repetition if the polymorphic form of the compound is not the selected polymorph.

16. The method of claim 15, wherein the cycle of irradiating the compound, assessing the polymorphic form, and repeating the crystallization with altered irradiation is repeated until crystallization results in the selected polymorph of the compound.

17. A method of assessing the polymorphic form of a substance in a microscopic field, the method comprising irradiating the substance, assessing Raman-shifted radiation scattered from the substance, and correlating the Raman-shifted radiation with the polymorphic form of the substance.

18. The method of claim 17, comprising assessing Raman-shifted radiation scattered from at least two different portions of the field.

19-49. (canceled)

50. A method of assessing the polymorphic form of a substance in a microscopic field, the method comprising identifying a portion of the field that contains the substance, irradiating the portion, assessing Raman-shifted radiation scattered from the portion, and correlating the Raman-shifted radiation with the polymorphic form of the substance.

51-54. (canceled)

55. A method of assessing the polymorphic form of a crystalline substance in a sample in a microscopic field, the method comprising irradiating the sample, assessing Raman-shifted radiation scattered from the sample, and correlating the Raman-shifted radiation with the polymorphic form or forms of the substance.

56-61. (canceled)

62. A device for generating a selected polymorph of a compound, the device comprising a temperature controller for thermally driving the compound through a phase transition and a radiation source for irradiating the compound with non-resonant radiation as the compound is thermally driven through the phase transition.

63. The device of claim 62, wherein the radiation source is a laser.

64. The device of claim 62, further comprising a substrate for supporting the compound as the as the compound is thermally driven through the phase transition.

65. The device of claim 62, further comprising a detector for detecting Raman-shifted radiation scattered by the compound.
